(19) United States
(12) Patent Application Publication
(10) Pub. No.: US 2003/0153618 A1
(43) Pub. Date: Aug. 14, 2003

(54) Treatment and prevention of herpes simplex infections

(76) Inventors: Larry Ford, Irvine, CA (US); Diane Ford, Irvine, CA (US)

Correspondence Address: McGuireWoods LLP, Suite 1800, 1750 Tysons Boulevard, McLean, VA, 22102, US

(21) Appl. No.: 10/266,928

(22) Filed: Oct. 9, 2002

Related U.S. Application Data
(63) Continuation of application No. 09/694,771, filed on Oct. 23, 2000, now abandoned.
(60) Provisional application No. 60/160,875, filed on Oct. 22, 1999.

(51) Int. Cl. A61K 31/198; A61K 31/22

(52) U.S. Cl. 514/551; 514/563; 514/564

(57) ABSTRACT

We have discovered that the topical application of the amino acid lysine and the 6-methyl derivative of lysine to Herpes simplex virus Type II is therapeutically beneficial and relieves the discomfort and symptoms of HSV infections and prevents or reduces the number of severity of HSV infections. We have also discovered that derivatives of lysine, in addition to 6-methyl lysine and derivatives of arginine have an inhibitory effect upon HSV replication. These compounds are useful in the prevention and treatment of HSV lesions.

### RELATED APPLICATION INFORMATION

This application claims priority from provisional application serial No. 60/160,875, filed Oct. 22, 1999.

### TECHNICAL FIELD

This invention relates to the treatment of infections of Herpes simplex viruses (HSV) and more particularly, to the treatment of genital infections of Herpes simplex viruses, i.e., Herpes simplex virus, Type II.

### BACKGROUND OF THE INVENTION

For a number of decades, it was believed that Herpes viruses were of only marginal clinical significance. It was generally agreed, for a long period of time, that the Herpes simplex virus (HSV) infections affected mainly children. It was also supposed that these infections produced only mild and self-limited diseases. Over the past two decades, however, this view has been radically changed and it has now been recognized that HSV are known to cause several severe, and sometime life-threatening, diseases in both children and adults. This recognition is the result of and has brought about increased study as to the ways in which HSV infections are spread and has resulted in a very intensive study of means for controlling the spread and treating these infections. It is now known, for example, that HSV infections in newborn infants are frequently devastating and may result in death or permanent injury to the central nervous system. Most HSV infections are Type II and are transmitted venereally or during delivery through a contaminated birth canal. Transmission of HSV from adults with non-genital lesions is less clearly understood but is important in instituting infection control.

More recently, the association of carcinoma of the cervix with viral infection has been well-demonstrated. While other diseases have been implicated by epidemiology and etiology, the association of classic Type II Herpes simplex virus vulvitis with vulvar carcinoma, and other association of Type II HSV infections with carcinoma of the cervix have been rather well documented.

In addition to these extremely serious and the life-threatening diseases, venereally transmitted HSV, Type II, have become a major concern in the treatment and prevention of venereal diseases generally.

It is also estimated that between fifty and seventy percent of all adults in the world are infected with HSV, Type I.

Many treatments and many compounds and preparations for the treatment of HSV infections have been proposed. The following are merely exemplary of the various approaches to the treatment of HSV infections which have been proposed: Topical therapy by 3% adenine arabinoside cream, 0.5% idoxuridine, photodynamic dyes, and diethyl ether have been reported as effective. (Lawrence Corey, et al., Medical Intelligence, Vol. 299, No. 5, pp. 237-239.) Among the photodynamic dyes which have been evaluated are proflaven, (Richard H. Kaufman, et al., Am. J. Obstet. Gynecol., Dec. 15, 1978, pp. 861-867; Neutral RedDye, Martin G. Myers, et al., New England Journal of Medicine, vol. 293, No. 19, pp. 945-949) and even inert solutions such as normal saline, has been used in conjunction with photoinactivation treatment. Other compounds which have been evaluated for effectiveness and efforts to control HSV infections include Nonoxynol 9 (L. A. Vontver, et al., Am. J. Obstet. Gynecol., Mar. 1, 1979, pp. 548-554) as well as other surfactants, e.g., quaternary ammonium cationic detergents. (Samuel S. Asculai, et al., Antimicrobial Agents and Chemotherapy, April 1978, pp. 686-690.) Chemical contraceptives have also been studied to determine their effect on HSV transmission and infections. (Balwant Singh, et al., Am. J. Obstet. Gynecol., Oct. 15, 1976, pp. 422-425; Bosko Postic, et al., Sexually Transmitted Diseases, January-March, 1978, pp. 22-24.) Phosphonacetic acid has been reported as being effective against Herpes virus in animals. (The Female Patient, April 1977, pp. 25-27.) There are, in addition, a number of preparations which are reported to have some therapeutic value in treating HSV infections. These include Ribavirin and Vidaravine.

### SUMMARY OF THE INVENTION

We have discovered that the topical application of the amino acid lysine and the 6-methyl derivative of lysine to Herpes simplex virus Type II is therapeutically beneficial and relieves the discomfort and symptoms of HSV infections and prevents or reduces the number and severity of HSV infections.

We have also discovered that derivatives of lysine, in addition to 6-methyl lysine and derivatives of arginine have an inhibitory effect upon HSV replication. These compounds are useful in the prevention and treatment of HSV lesions.

The compounds which are the subject of this invention include derivatives of lysine,

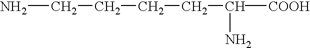

and derivatives of arginine

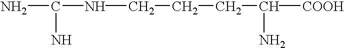

The derivatives which are specifically disclosed and claimed herein include the following:

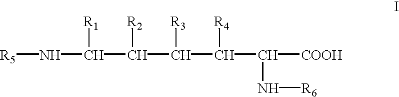

wherein none, one, two or three of R₁, R₂, R₃ and R₄ is methyl, hydroxyl, one to four carbon containing aliphatic ester, one to four carbon containing aliphatic ether and all others of said R₁, R₂, R₃ and R₄ are hydrogen, R₅ is hydrogen, methyl, ethyl, carbamyl, isopropyl, isopropenyl, hydroxyl, one to four carbon containing aliphatic ester, one to four carbon containing aliphatic ether, or acetimidoyl, and R₆ is hydrogen, methyl, acetyl, or carbamyl; or

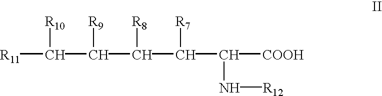

wherein none, one, two or three of R₇, R_{8,} R₉ and R₁₀ is methyl, hydroxyl, one to four carbon containing aliphatic ester, or one to four carbon containing aliphatic ether and all others of said R₇, R₈, R₉ and R₁₀ are hydrogen, R₁₁ is methyl, acetyl, isopropyl, isopropenyl, hydroxyl, one to four carbon containing aliphatic ester, or one to four carbon containing aliphatic ether, acetimidoyl, and R₁₂ is hydrogen, methyl, acetyl or carbamyl; or

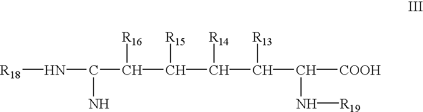

wherein none, one, two or three of R₁₃, R₁₄, R₁₅ and R₁₆ is methyl, hydroxyl, one to four carbon containing aliphatic ester, or one to four carbon containing aliphatic ether and none or one of R₁₃, R₁₄, R₁₅ and R₁₆ is imino, all others of said R₁₃, R₁₄, R₁₅ and R₁₆ hydrogen, and R₁₈ is hydrogen, methyl, acetyl or carbamyl; or

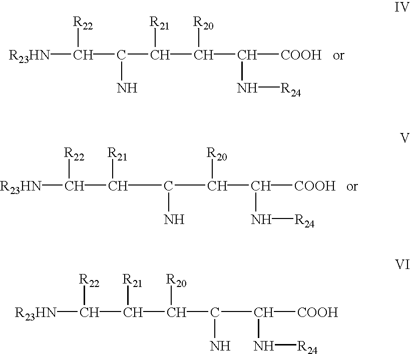

wherein none, one or two of R₂₀, R₂₁ and R₂₂ is methyl, hydroxyl, one to four carbon containing aliphatic ester, or one to four carbon containing aliphatic ether, all others of R₂₀, R₂₁ and R₂₂ being hydrogen, R₂₃ is hydrogen, methyl, acetyl, carbamyl, isopropyl, hydroxyl, one to four carbon containing aliphatic ester, or one to four carbon containing aliphatic ether or isopropenyl, and R₂₄ is hydrogen, methyl, acetyl or carbamyl; or

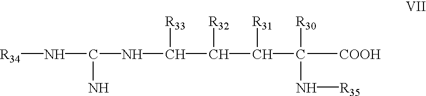

wherein none, one, two or three of R₃₀, R₃₂, R₃₂ and R₃₃ is methyl, hydroxyl, one to four carbon containing aliphatic ester, or one to four carbon containing aliphatic ether, or amino, all others of R₃₁, R₃₂, R₃₃ being hydrogen, R₃₄, is hydrogen, methyl, acetyl, amino, carbamyl, isopropyl, hydroxyl, one to four carbon containing aliphatic ester, or one to four carbon containing aliphatic ether, or isopropenyl, and R₃₅ is hydrogen, methyl, acetyl or carbamyl; or

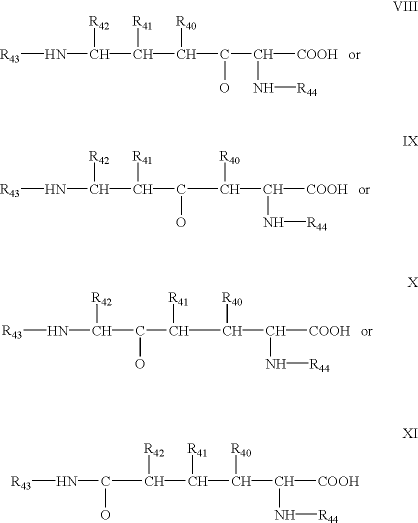

wherein none, one, two or three of R₄₀, R₄₁ and R₄₂ is methyl, hydroxyl, one to four carbon containing aliphatic ester, or one to four carbon containing aliphatic ether and all others of said R₄₀, R₄₁ and R₄₂ are hydrogen, R₄₃ is hydrogen, methyl, ethyl, carbamyl, isopropyl, isopropenyl, hydroxyl, one to four carbon containing aliphatic ether, or acetimidoyl, and R₄₄ is hydrogen, methyl, acetyl, or carbamyl.

### DESCRIPTION OF THE PREFERRED EMBODIMENTS

The method of this invention comprises the topical application of a composition in which the active ingredient for the treatment of Herpes simplex virus includes (e.g. consists essentially of) one or more of the aforesaid compounds or derivatives which are isomerically or otherwise equivalent thereof. This invention also comprises vaginal suppositories in which the active ingredient for the treatment of Herpes simplex virus, Type II, consist essentially of one or more of said compounds, alone or as mixtures thereof.

It will be understood that the composition of this invention will typically include, as a majority constituent, an inert material. For example, in solution, the inert materials will most commonly be water. Various emollients, buffers, viscosity controlling agents, preservatives, and stabilizing materials such as surfactants may be added, where desired. Suppositories will typically include, as a major constituent, a polyglycol or polyacrylamide compound which is solid at normal temperature but which melts or dissolves slowly at normal body temperature or which permits gradual extraction therefrom of the active agent. Insofar as the suppository carrier composition per se is concerned, any convenient suppository carrier material may be used. In addition, the compositions, e.g., creams, solutions or suppositories may include active ingredients for treating other conditions, so long as these active ingredients do not interfere with the action of the Herpes simplex virus treating compounds.

In addition, the composition for topical treatment of Herpes simplex virus may comprise a cream or an ointment, preferably, a cream or an ointment in which the active ingredient for the treatment of Herpes simplex virus is one of the aforesaid derivatives or a combination of the same. Such compositions comprise, as a major constituent, a water soluble carrier composition such as any of a large number of polyglycol compounds which are at least slightly soluble in water at body temperatures and which are in the form of a paste or highly viscous liquid at room temperature.

Other compositions using different bases and carriers may also be used within the scope of this invention. Indeed, any carrier or base which can be applied topically without interference with the activities of the anti-HSV active ingredient and which is sufficiently biologically compatible to permit topical application on infected or normal tissues may be used. Thus, a lipid base cream may be used, but is not preferred. All compatible bases for these compositions are within the cope and intent of this invention.

In general, the topical application composition of this invention may comprise any tissue compatible carrier containing an effective amount of one or more of the following derivatives of lysine or of arginine:

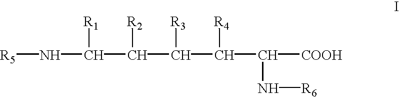

wherein none, one, two or three of R₁, R₂, R₃ and R₄ is methyl, hydroxyl, one to four carbon containing aliphatic ester, one to four carbon containing aliphatic ether and all others of said R₁, R₂, R₃ and R₄ are hydrogen. R₅ is hydrogen, methyl, ethyl, carbamyl, isopropyl, isopropenyl, hydroxyl, one to four carbon containing aliphatic ester, one to four carbon containing aliphatic ether, or acetimidoyl, and R₆ is hydrogen, methyl, acetyl, or carbamyl; or

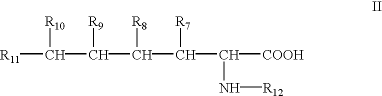

wherein none, one, two or three of R₇, R₈, R₉ and R₁₀ is methyl, hydroxyl, one to four carbon containing aliphatic ester, or one to four carbon containing aliphatic ether and all others of said R₇, R₈, R₉ and R₁₀ are hydrogen, R₁₁ is methyl, acetyl, isopropyl, isopropenyl, hydroxyl, one to four carbon containing aliphatic ester, or one to four carbon containing aliphatic ether, acetimidoyl, and R₁₂ is hydrogen, methyl, acetyl or carbamyl; or

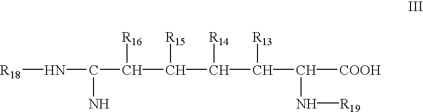

wherein none, one, two or three of R₁₃, R₁₄, R₁₅ and R₁₆ is methyl, hydroxyl, one to four carbon containing aliphatic ester, or one to four carbon containing aliphatic ether and none or one of R₁₃, R₁₄, R₁₅ and R₁₆ is imino, all others of said R₁₃, R₁₄, R₁₅ and R₁₆ hydrogen, and R₁₈ is hydrogen, methyl, acetyl or carbamyl; or

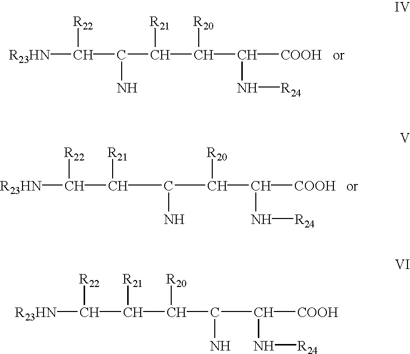

wherein none, one or two of R₂₀, R₂₁ and R₂₂ is methyl, hydroxyl, one to four carbon containing aliphatic ester, or one to four carbon containing aliphatic ether, all others of R₂₀, R₂₁ and R₂₂ being hydrogen, R₂₃ is hydrogen, methyl, acetyl, carbamyl, isopropyl, hydroxyl, one to four carbon containing aliphatic ester, or one to four carbon containing aliphatic ether or isopropenyl, and R₂₄ is hydrogen, methyl, acetyl or carbamyl; or

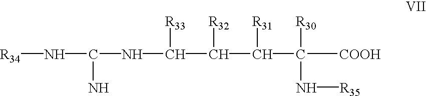

wherein none, one, two or three of R₃₀, R₃₂, R₃₂ and R₃₃ is methyl, hydroxyl, one to four carbon containing aliphatic ester, or one to four carbon containing aliphatic ether, or amino, all others of R₃₁, R₃₂, R₃₃ being hydrogen, R₃₄ is hydrogen, methyl, acetyl, amino, carbamyl, isopropyl, hydroxyl, one to four carbon containing aliphatic ester, or one to four carbon containing aliphatic ether, or isopropenyl, and R₃₅ is hydrogen, methyl, acetyl or carbamyl; or

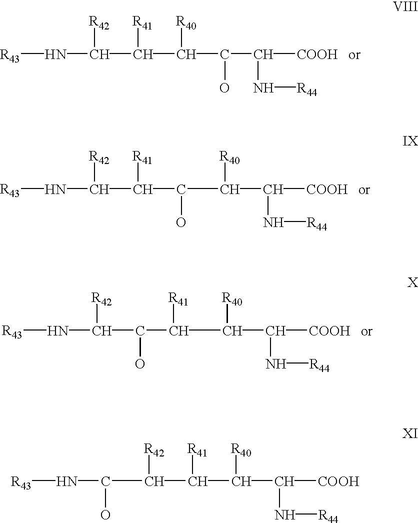

wherein none, one, two or three of R₄₀, R₄₁ and R₄₂ is methyl, hydroxyl, one to four carbon containing aliphatic ether and all others of said R₄₀, R₄₁ and R₄₂ are hydrogen, R₄₃ is hydrogen, methyl, ethyl, carbamyl, isopropyl, isopropenyl, hydroxyl, one to four carbon containing aliphatic ether, or acetimidoyl, and R₄₄ is hydrogen, methyl, acetyl, or carbamyl.

It is a matter of routine experimentation to determine the concentration above which no increased effectiveness is observed, as the higher end of the preferred range of concentration, and the concentration below which effectiveness begins to drop off, which will be the low end of the effective concentration range. If desired, higher concentrations could be used. The concentrations generally in the range of 0.05 to 5% by weight, are generally considered suitable, but concentrations as low as 0.01% and as high as several percent, e.g., saturated in the carrier, may be used.

The optimum concentration of the active ingredient will also be a function of the technique of application of a composition to the affected area. For example, a higher concentration may desirably be used in a douche which is applied twice a day, whereas it may be desirable to use a lower concentration of the active ingredient in capsules, ointments and suppositories where continuous contact, or long term contact, is contemplated.

Conventional compositions and preparative techniques and a wide range of concentrations may be used in preparing these compositions including the effective concentration of the active ingredient (e.g., douche solution, ointment, cream, or suppository, or other carrier material).

The scope of this invention is not limited to the examples, which are merely to demonstrate the effectiveness of the invention.

Initially, we had proved the correlation between in vitro test results in which the plaque-forming units were counted in media treated with various potentially inhibitory compounds and the efficacy of compounds which demonstrated significant inhibition of plaque formation in vivo treatment and prevention of lesions of HSV Type II infections.

Absorbed virons were absorbed in susceptible cells in chemically defined media and the number of plaques made were counted. (See Davis, B. D., et al., Microbiology, 2^{nd} Ed., Harper & Row, 1973, Part V, and sources cited therein for procedural details and discussion of the plaque method.)

The cell line chosen was green monkey kidney cells (BSC) that have been passed greater than 41 times. HSV, Type II, stock virons that had exhibited four-plus cytopathic effect on cells had been denatured by three times freeze-thaw followed by centrifugation at 5,000×g for 10 minutes at 4° C. The virons were added to a confluent lawn of BSC cells and allowed to absorb at 25° C. for one hour. The unabsorbed virons and the media were removed by washing three times in PBS. The cells were then covered with defined media with a lysine/arginine ratio of 500:1. The control media contained a lysine/arginine ratio of 1:1. The plaques were counted when easily readable. Flasks are done in triplicate.

TABLE A

| Experiment | Lysine: | Plaque Forming | Unit Count |
|---|---|---|---|
| No. | Arginine Ratio | 500:1 | 1:1 |
| 1 | | 7 | 594 |
| 2 | | 6 | 631 |
| 3 | | 11 | 621 |
| 4 | | 23 | 936 |
| 5 | | 17 | 781 |
| 6 | | 5 | 814 |
| | Average | 12 | 738 |

Once the efficacy of lysine and arginine competition was well demonstrated in vitro, the lysine-arginine data were rerun as a control and derivatives of lysine and arginine were made and tested in a similar fashion but at different concentrations and ratios.

TABLE B

| | P.F.U. | Count* |
|---|---|---|
| Compound Ratio to Arginine → | 10:1 | 100:1 |
| 6-Methyl lysine | <5 | <5 |
| 4-Methyl lysine | 541 | 140 |
| 5-Methyl lysine | 79 | 43 |
| 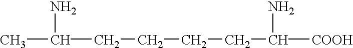 | <5 | <5 |
| 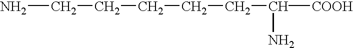 | <5 | <5 |

*Average of three tests.
All of the preceding were done by direct PFU, plaque-forming unit, counts.

### Back Titration Experiments

The following experiments were conducted by back titration:

Herpes simplex II virus from laboratory stocks was used.

BSC cells were grown to confluence in Eagles medium with 15% fetal calf serum, 150 units per milliliter of penicillin and 100 micrograms per milliliter of streptomycin. The cultures were inoculated with 500 PFU of HS II. The virons were allowed to absorb for one hour at 25° C. The tissue cultures were then washed three times with sterile PBS to remove excess media and non-absorbed virons. The cells were then incubated at 37° C. until four-plus cytopathic effects were observed. The cells were then disrupted by slow freeze-thaw two times. The suspension was then clarified by slow speed centrifugation at 4° C. The supemanant suspension was then added to confluent BSC cultures. After one hour absorption, the cells were washed with PBS twice. The cells were overlaid in defined reagent (in triplicate) Eagles media in noble agar. The cells were then incubated at 37° C. until the plaques were easily counted, usually 40 t 72 hours. the cells were then washed two times with PBS and then fixed with 10% H₂CO and a 2% sodium acetate. The cells were then stained in 0.1% crystal violet in ethanol.

Although the results indicate that in increasing order of blocking the arginine molecule, 6-methyl lysine was the third most efficacious, it was by far the easiest to synthesize and thus was used for the first clinical trials.

In the tables which follow the concentration of the compound to be tested, in micrograms per milliliter, is listed across the top of the table and the concentration of arginine in micrograms per milliliter is listed down the left side of the table, the count of plaque-forming units being shown in the matrix.

TABLE C*
Compound under Test:

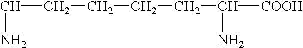

| μg/ml | μg/ml of Compound under test | | | | | | | | |
|---|---|---|---|---|---|---|---|---|---|
| Arginine | 0 | 10 | 25 | 50 | 100 | 200 | 300 | 400 | 500 |
| 0 | 23 | 0 | 0 | 0 | 0 | 0 | 0 | 0 | 0 |
| 2.5 | 4 × 10³ | 0 | 0 | 0 | 0 | 0 | 0 | 0 | 0 |
| 5.0 | 3.9 × 10⁴ | 0 | 0 | 0 | 0 | 0 | 0 | 0 | 0 |
| 7.5 | 6.7 × 10⁴ | 0 | 0 | 0 | 0 | 0 | 0 | 0 | 0 |
| 10 | 9.1 × 10⁴ | 290 | 3.7 × 10³ | 79 | 0 | 0 | 0 | 0 | 0 |
| 15 | 2.3 × 10⁵ | 799 | 4.2 × 10³ | 276 | 0 | 0 | 0 | 0 | 0 |
| 20 | 2.8 × 10⁵ | 2.8 × 10⁵ | 2.6 × 10⁵ | 6.6 × 10⁴ | 3.2 × 10³ | 68 | 0 | 0 | 0 |
| 25 | 3.1 × 10⁵ | 2.8 × 10⁵ | 4 × 10⁵ | 3.6 × 10⁴ | 6.6 × 10³ | 654 | 0 | 0 | 0 |

*Matrix values are P.F.U. Counts for composition having the stated amount of the compound under test and arginine in the stated amount.

TABLE D*
Compound under Test:

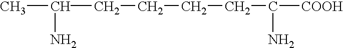

| μg/ml | μg/ml of Compound under test | | | | | | | | |
|---|---|---|---|---|---|---|---|---|---|
| Arginine | 0 | 10 | 25 | 50 | 100 | 200 | 300 | 400 | 500 |
| 0 | 41 | 0 | 0 | 0 | 0 | 0 | 0 | 0 | 0 |
| 2.5 | 4.1 × 10³ | 0 | 0 | 0 | 0 | 0 | 0 | 0 | 0 |
| 5.0 | 3.9 × 10⁷ | 0 | 0 | 0 | 0 | 0 | 0 | 0 | 0 |
| 7.5 | 6.9 × 10⁴ | 0 | 0 | 0 | 0 | 0 | 0 | 0 | 0 |
| 10 | 9.1 × 10⁴ | 610 | 3.7 × 10³ | 79 | 0 | 0 | 0 | 0 | 0 |
| 15 | 2.3 × 10⁵ | 46 | 4.2 × 10³ | 276 | 0 | 0 | 0 | 179 | 0 |
| 20 | 2.9 × 10⁵ | 2.1 × 10⁵ | 2.0 × 10⁵ | 1.6 × 10⁴ | 1.0 × 10³ | 760 | 163 | 310 | 0 |
| 25 | 3.3 × 10⁵ | 2.8 × 10⁵ | 2.1 × 10⁵ | 1.1 × 10⁴ | 1.6 × 10³ | 1.7 × 10³ | 1.0 × 10³ | 140 | 0 |

*Matrix values are P.F.U. Counts for composition having the stated amount of the compound under test and arginine in the stated amount.

TABLE E*
Compound under Test: 6-Methyl lysine

| μg/ml | μg/ml of Compound under test | | | | | | | | |
|---|---|---|---|---|---|---|---|---|---|
| Arginine | 0 | 10 | 25 | 50 | 100 | 200 | 300 | 400 | 500 |
| 0 | 16 | 0 | 0 | 0 | 0 | 0 | 0 | 0 | 0 |
| 2.5 | 4.1 × 10³ | 0 | 0 | 0 | 0 | 0 | 0 | 0 | 0 |
| 5.0 | 3.7 × 10⁴ | 0 | 0 | 0 | 0 | 0 | 0 | 0 | 0 |
| 7.5 | 6.5 × 10⁴ | 0 | 0 | 0 | 0 | 0 | 0 | 0 | 0 |
| 10 | 9.1 × 10⁴ | 4.0 × 10³ | 2.1 × 10³ | 0 | 0 | 0 | 0 | 0 | 0 |
| 15 | 2.2 × 10⁵ | 1.8 × 10³ | 2.1 × 10³ | 1.7 × 10³ | 0 | 0 | 0 | 0 | 0 |
| 20 | 2.9 × 10⁵ | 2.8 × 10⁶ | 2.4 × 10⁵ | 1.7 × 10⁵ | 6.4 × 10⁴ | 6.9 × 10⁴ | 5.0 × 10³ | 690 | 56 |
| 25 | 3.0 × 10⁵ | 2.7 × 10⁵ | 2.9 × 10⁵ | 2.0 × 10⁵ | 5.8 × 10⁴ | 4.7 × 10⁴ | 4.0 × 10⁴ | 3.9 × 10³ | 3.71 × 10³ |

*Matrix values are P.F.U. Counts for composition having the stated amount of the compound under test and arginine in the stated amount.

TABLE F*
Compound under Test: 5-Methyl lysine

| μg/ml | μg/ml of Compound under test | | | | | | | | |
|---|---|---|---|---|---|---|---|---|---|
| Arginine | 0 | 10 | 25 | 50 | 100 | 200 | 300 | 400 | 500 |
| 0 | 21 | 30 | 0 | 0 | 0 | 0 | 0 | 0 | 0 |
| 2.5 | 4.2 × 10³ | 2.7 × 10³ | 697 | 0 | 0 | 0 | 0 | 0 | 0 |
| 5.0 | 3.7 × 10⁴ | 3.7 × 10⁴ | 1.4 × 10³ | 190 | 69 | 0 | 0 | 0 | 0 |
| 7.5 | 6.5 × 10⁴ | 6.0 × 10⁴ | 4.7 × 10³ | 190 | 170 | 0 | 0 | 0 | 0 |
| 10 | 8.2 × 10⁴ | 7.9 × 10⁴ | 6.5 × 10⁴ | 1.7 × 10³ | 656 | 79 | 0 | 0 | 0 |
| 15 | 2.2 × 10⁵ | 2.0 × 10⁵ | 1.7 × 10⁴ | 1.3 × 10³ | 710 | 63 | 1.9 × 10⁴ | 1.9 × 10⁴ | 1.0 × 10⁴ |
| 20 | 2.7 × 10⁵ | 3.0 × 10⁵ | 3.1 × 10⁵ | 3.0 × 10⁵ | 1.2 × 10⁵ | 2.7 × 10⁵ | 5.6 × 10⁴ | 1.7 × 10⁴ | 2.3 × 10³ |
| 25 | 3.2 × 10⁵ | 3.0 × 10⁵ | 3.0 × 10⁵ | 3.0 × 10⁵ | 2.4 × 10⁵ | 4.1 × 10⁵ | 6.1 × 10⁴ | 4.6 × 10⁴ | 4.0 × 10⁴ |

*Matrix values are P.F.U. Counts for composition having the stated amount of the compound under test and arginine in the stated amount.

TABLE G*
Compound under Test: 4-Methyl lysine

| μg/ml | μg/ml of Compound under test | | | | | | | | |
|---|---|---|---|---|---|---|---|---|---|
| Arginine | 0 | 10 | 25 | 50 | 100 | 200 | 300 | 400 | 500 |
| 0 | 61 | 37 | 10 | 0 | 0 | 0 | 0 | 0 | 0 |
| 2.5 | 4.0 × 10³ | 110 | 74 | 0 | 0 | 0 | 0 | 0 | 0 |
| 5.0 | 4.1 × 10⁴ | 3.7 × 10³ | 310 | 671 | 371 | 191 | 169 | 671 | 239 |
| 7.5 | 7.1 × 10⁴ | 7.2 × 10⁴ | 6.6 × 10⁴ | 5.0 × 10⁴ | 4.2 × 10⁴ | 1.0 × 10⁴ | 1.1 × 10⁷ | 1.9 × 10³ | 6.7 × 10³ |
| 10 | 9.4 × 10⁴ | 9.6 × 10⁴ | 8.7 × 10⁴ | 7.0 × 10⁴ | 4.0 × 10⁷ | 1.8 × 10⁴ | 1.9 × 10⁴ | 1.7 × 10⁴ | 1.3 × 10⁴ |
| 15 | 2.3 × 10⁵ | 2.3 × 10⁵ | 9.6 × 10⁴ | 7.9 × 10⁴ | 5.0 × 10⁴ | 6.8 × 10⁴ | 2.7 × 10⁴ | 2.0 × 10⁴ | 1.4 × 10⁴ |
| 20 | 3.1 × 10⁵ | 3.1 × 10⁵ | 3.3 × 10⁵ | 3.1 × 10⁵ | 3.6 × 10⁵ | 3.2 × 10⁵ | 3.1 × 10⁵ | 3.0 × 10⁵ | 2.4 × 10⁵ |
| 25 | 3.2 × 10⁵ | 3.3 × 10⁵ | 3.4 × 10⁵ | 3.6 × 10⁵ | 3.1 × 10⁵ | 3.0 × 10⁵ | 3.3 × 10⁵ | 3.3 × 10⁵ | 2.9 × 10⁵ |

*Matrix values are P.F.U. Counts for composition having the stated amount of the compound under test and arginine in the stated amount.

It is emphasized that the data specifically set forth are exemplary only, and these data do not limit the invention which is the subject of this application, namely, the methods of treating Herpes simplex virus, Type II, by direct or topical application, to the sites of the infection or expected infection, of a composition which comprises a major proportion of carrier, e.g., an inert liquid, ointment or suppository carrier (which may include other active ingredients, preservatives, etc. which do not interfere with the present invention) containing an effective amount, e.g., generally in the range of from about 0.005% to 5 percent or higher, even up to saturation, of an active constituent or constituents consisting essentially of one or more of the aforesaid compounds:

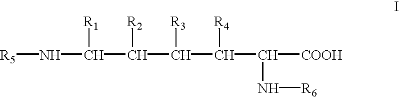

wherein none, one, two or three of R₁, R₂, R₃ and R₄ is methyl, hydroxyl, one to four carbon containing aliphatic ester, one to four carbon containing aliphatic ether and al others of said R₁, R₂, R₃ and R₄ are hydrogen, R₅ is hydrogen, methyl, ethyl, carbamyl, isopropyl, isopropenyl, hydroxyl, one to four carbon containing. aliphatic ester, one to four carbon containing aliphatic ether, or acetimidoyl, and R₆ is hydrogen, methyl, acetyl, or carbamyl; or

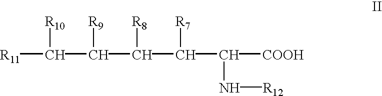

wherein none, one, two or three of R₇, R₈, R₉ and R₁₀ in methyl, hydroxyl, one to four carbon containing aliphatic ester, or one to four carbon containing aliphatic ether and all others of said R₇, R₈, R₉ and R₁₀ are hydrogen, R₁₁ is methyl, acetyl, isopropyl, isopropenyl, hydroxyl, one to four carbon containing aliphatic ester, or one to four carbon containing aliphatic ether, acetimidoyl, and R₁₂ is hydrogen, methyl, acetyl or carbamyl; or

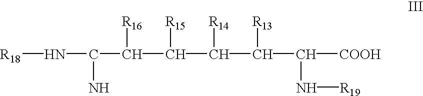

wherein none, one, two or three of R₁₃, R₁₄, R₁₅ and R₁₆ is methyl, hydroxyl, one to four carbon containing aliphatic ester, or one to four carbon containing aliphatic ether and none or one of R₁₃, R₁₄, R₁₅ and R₁₆ is imino, all others of said R₁₃, R₁₄, R₁₅ and R₁₆ being hydrogen, and R₁₈ is hydrogen, methyl, acetyl or carbamyl; or

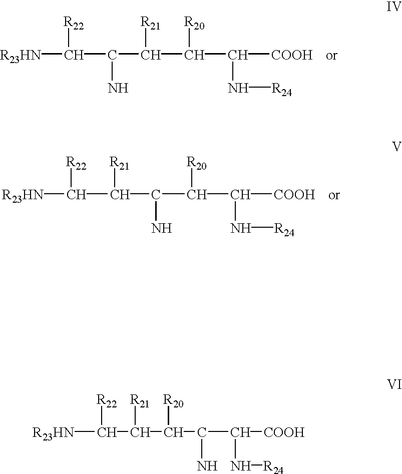

wherein none, one or two of R₂₀, R₂₁ and R₂₂ is methyl, hydroxyl, one to four carbon containing aliphatic ester or one to four carbon containing aliphatic ether, all others of R₂₀, R₂₁ and R₂₂ being hydrogen, R₂₃ is hydrogen, methyl, acetyl, carbamyl, isopropyl, hydroxyl, one to four carbon containing aliphatic ester, or one to four carbon containing aliphatic ether or isopropenyl, and R₂₄ is hydrogen, methyl, acetyl or carbamyl; or

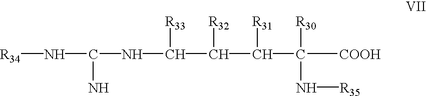

wherein none, one, two or three of R₃₀, R₃₂, R₃₂ and R₃₃ is methyl, hydroxyl, one to four carbon containing aliphatic ester, or one to four carbon containing aliphatic ether, or amino, all others of R₃₁, R₃₂, R₃₃ being hydrogen, R₃₄ is hydrogen, methyl, acetyl, amino, carbamyl, isopropyl, hydroxyl, one to four carbon containing aliphatic ester, or one to four carbon containing aliphatic ether, or isopropenyl, and R₃₅ is hydrogen, methyl, acetyl or carbamyl; or

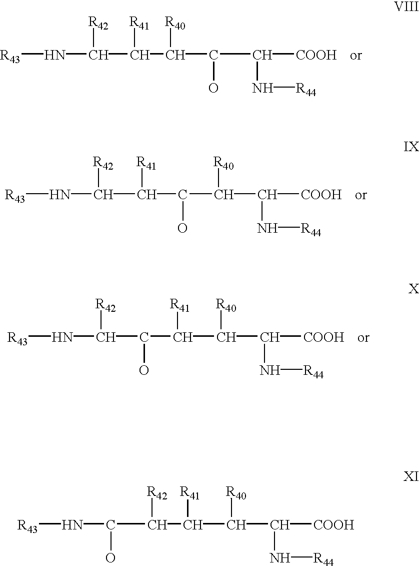

wherein none, one, two or three of R₄₀, R₄₁ and R₄₂ is methyl, hydroxyl, one to four carbon containing aliphatic ester, or one to four carbon containing aliphatic ether and all others of said R₄₀, R₄₁ and R₄₂ are hydrogen, R₄₃ is hydrogen, methyl, ethyl, carbamyl, isopropyl, isopropenyl, hydroxyl, one to four carbon containing aliphatic ether, or acetimidoyl, and R₄₄ is hydrogen, methyl, acetyl, or carbamyl.

wherein none, one, two or three of R₁₃, R₁₄, R₁₅ and R₁₆ is methyl, hydroxyl, one to four carbon containing aliphatic ester, or one to four carbon containing aliphatic ether and none or one of R₁₃, R₁₄, R₁₅ and R₁₆ is imino, all others of said R₁₃, R₁₄, R₁₅ and R₁₆ being hydrogen, and R₁₈ is hydrogen, methyl, acetyl or carbamyl; or

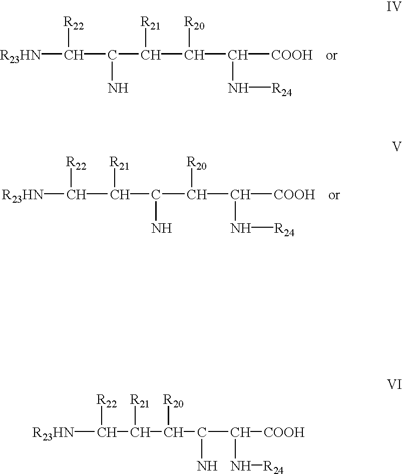

wherein none, one or two of R₂₀, R₂₁ and R₂₂ is methyl, hydroxyl, one to four carbon containing aliphatic ester, or one to four carbon containing aliphatic ether, all others of R₂₀, R₂₁ and R₂₂ being hydrogen, R₂₃ is hydrogen, methyl, acetyl, carbamyl, isopropyl, hydroxyl, one to four carbon containing aliphatic ester, or one to four carbon containing aliphatic ether or isopropenyl, and R₂₄ is hydrogen, methyl, acetyl or carbamyl; or

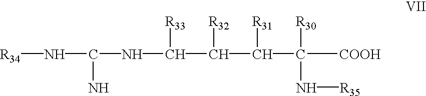

wherein none, one, two or three of R₃₀, R₃₂, R₃₂ and R₃₃ is methyl, hydroxyl, one to four carbon containing aliphatic ester, or one to four carbon containing alipathic ether, or amino, all others of R₃₁, R₃₂, R₃₃ being hydrogen, R₃₄ is hydrogen, methyl, acetyl, amino, carbamyl, isopropyl, hydroxyl, one to four carbon containing aliphatic ester or one to four carbon containing aliphatic ether or isopropenyl, and R₃₅ is hydrogen, methyl, acetyl or carbamyl; or

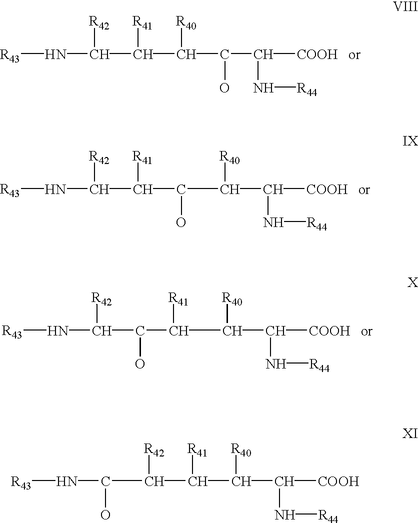

wherein none, one, two or three of R₄₀, R₄₁ and R₄₂ is methyl, hydroxyl, one to four carbon containing aliphatic ester, or one to four carbon containing aliphatic ether and all others of said R₄₀, R₄₁ and R₄₂ are hydrogen, R₄₃ is hydrogen, methyl, ethyl, carbamyl, isopropyl, isopropenyl, hydroxyl, one to four carbon containing aliphatic ether, or acetimidoyl, and R₄₄ is hydrogen, methyl, acetyl, or carbamyl.

wherein none, one, two or three of R₁₃, R₁₄, R₁₅ and R₁₆ is methyl, hydroxyl, one to four carbon containing aliphatic ester, or one to four carbon containing aliphatic ether and none or one of R₁₃, R₁₄, R₁₅ and R₁₆ is imino, all others of said R₁₃, R₁₄, R₁₅ and R₁₆ being hydrogen, and R₁₈ is hydrogen, methyl, acetyl or carbamyl; or

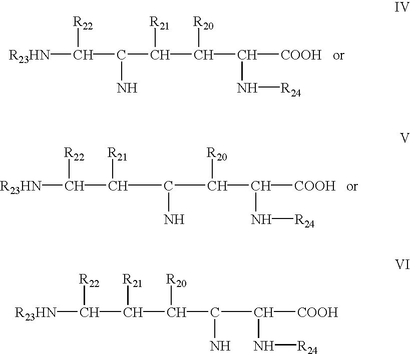

wherein none, one or two of R₂₀, R₂₁ and R₂₂ is methyl, hydroxyl, one to four carbon containing aliphatic ester, or one to four carbon containing aliphatic ether, all others of R₂₀, R₂₁ and R₂₂ being hydrogen, R₂₃ is hydrogen, methyl, acetyl, carbamyl, isopropyl, hydroxyl, one to four carbon containing aliphatic ether or isopropenyl, and R₂₄ is hydrogen, methyl, acetyl or carbamyl; or

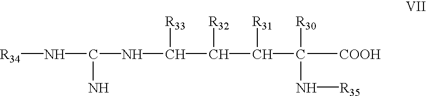

wherein none, one, two or three of R₃₀, R₃₂, R₃₂ and R₃₃ is methyl, hydroxyl, one to four carbon containing aliphatic ester, or one to four carbon containing aliphatic ether, or amino, all others of R₃₁, R₃₂, R₃₃ being hydrogen, R₃₄ is hydrogen, methyl, acetyl, amino, carbamyl, isopropyl, hydroxyl, one to four carbon containing aliphatic ester or one to four carbon containing aliphatic ether or isopropenyl, and R₃₅ is hydrogen, methyl, acetyl or carbamyl; or

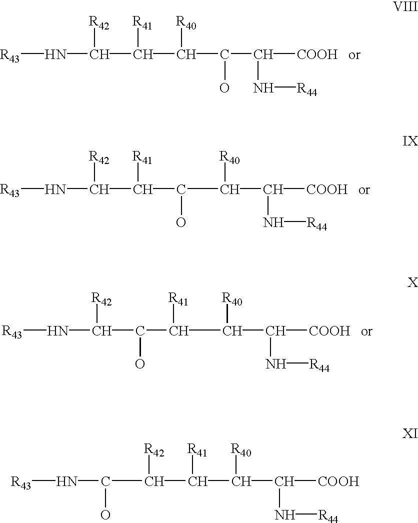

wherein none, one, two or three of R₄₀, R₄₁ and R₄₂ is methyl, hydroxyl, one to four carbon containing aliphatic ester, or one to four carbon containing aliphatic ether and all others of said R₄₀, R₄₁ and R₄₂ are hydrogen, R₄₃ is hydrogen, methyl, ethyl, carbamyl, isopropyl, isopropenyl, hydroxyl, one to four carbon containing aliphatic ether, or acetimidoyl, and R₄₄ is hydrogen, methyl, acetyl, or carbamyl.

What is claimed is:

1. Chemical compounds combinations suitable for treatment of vaginal herpes simplex disease comprising:
a physically inert carrier suppository composition which is solid at room temperature and fluid at human body temperature; and
a 0.01 to 5.0 wt % concentration of an amino acid selected from the class consisting of 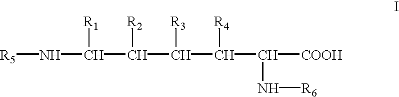
wherein none, one, two or three of R₁, R₂, R₃ and R₄ is methyl, hydroxyl, one to four carbon containing aliphatic ester, one to four carbon containing aliphatic ether and all others of said R₁, R₂, R₃ and R₄ are hydrogen, R₅ is hydrogen, methyl, ethyl, carbamyl, isopropyl, isopropenyl, hydroxyl, one to four carbon containing aliphatic ester, one to four carbon containing aliphatic ether, or acetimidoyl, and R₆ is hydrogen, methyl, acetyl, or carbamyl; or 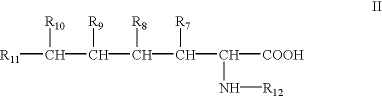
wherein none, one, two or three of R₇, R_{8,} R₉ and R₁₀ is methyl, hydroxyl, one to four carbon containing aliphatic ester, or one to four carbon containing aliphatic ether and all others of said R₇, R₈, R₉ and R₁₀ are hydrogen, R₁₁ ism ethyl, acetyl, isopropyl, isopropenyl, hydroxyl, one to four carbon containing aliphatic ester, or one to four carbon containing aliphatic ether, acetimidoyl, and R₁₂ is hydrogen, methyl, acetyl or carbamyl, or

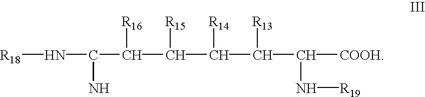

2. Chemical compound combination suitable for treatment of herpes simplex disease comprising:
a water soluble physiologically inert compatible base, and
a 0.01 to 5.0 wt % concentration of an amino acid selected from the class of consisting of 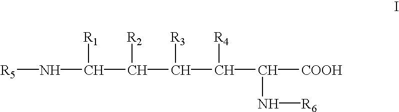
wherein none, one, two or three of R₁, R₂, R₃ and R₄ is methyl, hydroxyl, one to four carbon containing aliphatic ester, one to four carbon containing aliphatic ether and all others of said R₁, R₂, R₃ and R₄ are hydrogen, R₅ is hydrogen, methyl, ethyl, carbamyl, isopropyl, isopropenyl, hydroxyl, one to four carbon containing aliphatic ester, one to four carbon containing aliphatic ether, or acetimidoyl, and R₆ is hydrogen, methyl, acetyl, or carbamyl; or 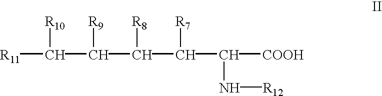
wherein none, one, two or three of R₇, R₈, R₉ and R₁₀ is methyl, hydroxyl, one to four carbon containing aliphatic ester, or one to four carbon containing aliphatic ether and all others of said R₇, R₈, R₉ and R₁₀ are hydrogen, R₁₁ is methyl, acetyl, isopropyl, isopropenyl, hydroxyl, one to four carbon containing aliphatic ether, acetimidoyl, and R₁₂ is hydrogen, methyl, acetyl or carbamyl; or

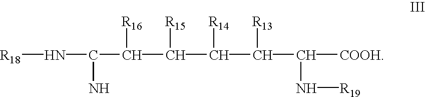

3. A process for treating herpes simplex disease infection of humans comprising:
applying a composition of a water soluble physiologically inert compatible base; and
a 0.01 to 5.0 wt % concentration of an amino acid selected from the class consisting of 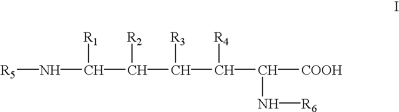
wherein none, one, two or three of R₁, R₂, R₃ and R₄ is methyl, hydroxyl, one to four carbon containing aliphatic ester, one to four carbon containing aliphatic ether and all others of said R₁, R₂, R₃ and R₄ are hydrogen, R₅ is hydrogen, methyl, ethyl, carbamyl, isopropyl, isopropenyl, hydroxyl, one to four carbon containing aliphatic ester, one to four carbon containing aliphatic ether, or acetimidoyl, and R₆ is hydroge n, methyl, acetyl, or carbamyl; or 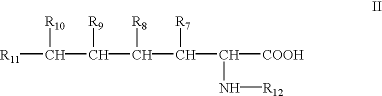
wherein none, one, two or three of R₇, R₈, R₉ and R₁₀ is methyl, hydroxyl, one to fur carbon containing aliphatic ester, or one to four carbon containing aliphatic ether and all others of said R₇, R_{8,} R₉ and R₁₀ are hydrogen, R₁₁ is methyl, acetyl, isopropyl, isopropenyl, hydroxyl, one to four carbon containing aliphatic ester, or one to four carbon containing aliphatic ether, acetimidoyl, and R₁₂ is hydrogen, methyl, acetyl or carbamyl; or 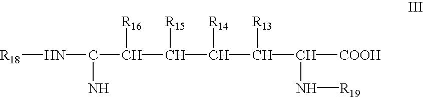
wherein none, one, two or three of R₁₃, R₁₄, R₁₅ and R₁₆ is methyl, hydroxyl, one to four carbon containing aliphatic ester, or one to four carbon containing aliphatic ether and none or one of R₁₃, R₁₄, R₁₅ and R₁₆ is imino, all others of said R₁₃, R₁₄, R₁₅ and R₁₆ being hydrogen, and R₁₈ is hydrogen, methyl, acetyl or carbamyl; or 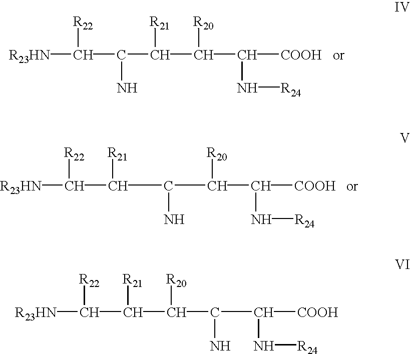
wherein none, one or two of R₂₀, R₂₁ and R₂₂ is methyl, hydroxyl, one to four carbon containing aliphatic ester, or one to four carbon containing aliphatic ether, all others of R₂₀, R₂₁ and R₂₂ being hydrogen, R₂₃ is hydrogen, methyl, acetyl, carbamyl, isopropyl, hydroxyl, one to four carbon containing aliphatic ester, or one to four carbon containing aliphatic ether or isopropenyl, and R₂₄ is hydrogen, methyl, acetyl or carbamyl; or 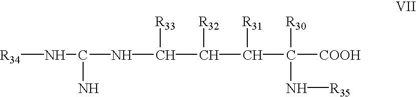
wherein none, one, two or three of R₃₀, R₃₂ and R₃₃ is methyl, hydroxyl, one to four carbon containing aliphatic ester, or one to four carbon containing aliphatic ether, or amino, all others of R₃₁, R₃₂, R₃₃ being hydrogen, R₃₄ is hydrogen, methyl, acetyl, amino, carbamyl, isopropyl, hydroxyl, one to four carbon containing aliphatic ester, or one to four carbon containing aliphatic ether, or isopropenyl, and R₃₅ is hydrogen, methyl, acetyl or carbamyl; or 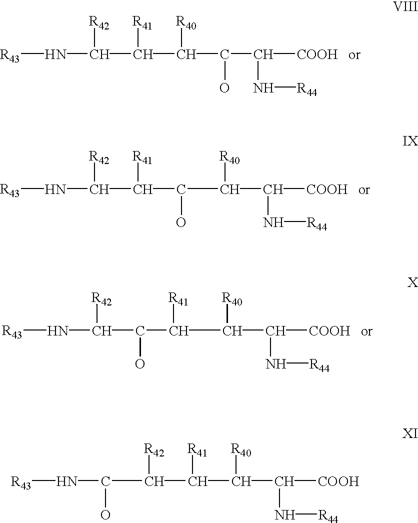
wherein none, one, two or three of R₄₀, R₄₁ and R₄₂ is methyl, hydroxyl, one to four carbon containing aliphatic ester, or one to four carbon containing aliphatic ether and all others of said R₄₀, R₄₁ and R₄₂ are hydrogen, R₄₃ is hydrogen, methyl, ethyl, carbamyl, isopropyl, isopropenyl, hydroxyl, one to four carbon containing aliphatic ether, or acetimidoyl, and R₄₄ is hydrogen, methyl, acetyl, or carbamyl.
